(19) United States
(12) Patent Application Publication
(10) Pub. No.: US 2026/0047860 A1
(43) Pub. Date: Feb. 19, 2026

(54) GROOVED COLD SNARE

(71) Applicant: COOK MEDICAL TECHNOLOGIES LLC, Bloomington, IN (US)

(72) Inventors: Evan Creaven, Ballina (IE); Kevin Doyle, Castletroy (IE); Ciara Kavanagh, Surry Hills (AU); Cathal Locke, Bronte (AU); Cathal McGrath, Oranmore (IE); Isadora Oliveira, Ennis (IE)

(21) Appl. No.: 19/285,318

(22) Filed: Jul. 30, 2025

Related U.S. Application Data
(60) Provisional application No. 63/683,388, filed on Aug. 15, 2024.

(51) Int. Cl. A61B 17/3205

(52) U.S. Cl. CPC A61B 17/32056

(57) ABSTRACT

A snare including a snare wire. The snare wire is formed into a loop, includes an exterior surface, and exhibits a length and a cross-section. The cross-section is orthogonal to the length. The snare wire includes a groove extending helically along the length of the snare wire. The groove is defined by an open channel including an opening defined at the exterior surface. The cross-section defines a center line and a cross-sectional length. The open channel exhibits a groove depth less than half of the cross-sectional length. The open channel is bound in the cross-section by a first line and a second line, the first line intersects the first point and the second line intersects the second point, the first line and the second line are parallel to the center line, and the first line and the second line are equidistant from the center line.

### CROSS-REFERENCE TO RELATED APPLICATIONS

The present application claims the benefit of U.S. Provisional Application No. 63/683,388, filed on Aug. 15, 2024, the teachings of which are incorporated by reference.

### FIELD

The present disclosure relates to medical devices and particularly to a snare including a grooved cold snare wire, a method of assembling the snare, and a method for resecting a polyp with the snare.

### BACKGROUND

Polyps grow from the mucosa of nasal passages, the gastrointestinal tract, the uterus, and other hollow organs that include a cavity or passage within the organ. Polyps are generally formed from an overgrowth of cells in these regions. In some instances, polyps may form tumors that could become cancerous. Polyps may be flat, dome shaped (sessile), or pendant from a stalk (pedunculated) that extends from the mucosal lining. The size may vary from a few millimeters to over a centimeter.

Endoscopic procedures allow for the observation of mucosal linings and the detection and removal of polyps that may be present. A common endoscopic procedure is the colonoscopy. During a colonoscopy, the inner lining of the colon is observed and polyps, if present, are removed. Endoscopic procedures are performed using an endoscope, which includes a light for illuminating tissue and a camera for capturing images or video of the illuminated tissue. An endoscope may also include one or more ports through which various devices, such as a polypectomy snare or basket, may be introduced. Polypectomy snares are used to capture and resect polyps. Either a cold snare or a hot (electrocautery) snare may be used depending on the size of the polyp, the shape of the polyp, and whether the polyp is cancerous.

A snare includes a snare wire that is formed into a retractable loop. The loop is typically positioned around the base of polyp and retracted until the polyp is grasped and resected by the loop. While grasping the polyp, care is taken to prevent the loop from slipping and resecting less than the desired amount of the polyp. If less than the desired amount of the polyp is removed, the polyp may return.

Thus, while present snares and snare wires achieve their intended purpose, there is a need for new and improved snares for resecting polyps from mucosal linings in the body.

### SUMMARY

According to various aspects, the present disclosure relates to a snare. The snare includes a sheath defining a lumen. The sheath includes a proximal end and a distal end. The snare also includes a shaft slidably disposed in the sheath and a snare wire coupled to the shaft. The snare wire is formed into a loop, includes an exterior surface, and exhibits a length and a cross-section, wherein the cross-section is orthogonal to the length. The snare wire also includes a groove extending helically along the length of the snare wire. The groove is defined by an open channel in the cross-section, the open channel includes an opening defined at the exterior surface between a first point and a second point. In addition, the cross-section defines a center line and a cross-sectional length. The open channel exhibits a groove depth less than half of the cross-sectional length, and the open channel is bound in the cross-section by a first line and a second line, the first line intersects the first point and the second line intersects the second point, the first line and the second line are parallel to the center line, and the first line and the second line are equidistant from the center line.

In embodiments of the above, the cross-section exhibits a cross-sectional diameter in the range of 0.2 millimeters to 0.8 millimeters.

In any of the above embodiments, the groove defines a helix pitch along the length of the snare wire and the helix pitch is in the range of 2 millimeters to 3 millimeters.

In any of the above embodiments, the cross-section defines a total cross-sectional area and the open channel defines a second cross-sectional area overlapping the total cross-sectional area, wherein the second cross-sectional area is in the range of 5 percent to 20 percent of the total cross-sectional area.

In any of the above embodiments, the open channel exhibits a geometry that is mirrored around the center line.

In any of the above embodiments, the groove width is in the range of 0.05 millimeters to 1.5 millimeters.

In any of the above embodiments, the groove depth is in the range of 0.04 millimeters to 0.06 millimeters.

In any of the above embodiments, the cross-section of the groove is concave and exhibits groove diameter in the range of 0.2 millimeters to 0.3 millimeters.

In any of the above embodiments, the exterior surface and a first surface of the open channel adjacent to the exterior surface meet at the first point at an acute angle and the exterior surface and a second surface of the open channel adjacent to the exterior surface meet at the second point at an acute angle.

In any of the above embodiments, the open channel includes a bottom and wherein an angle in the range of 45 degrees to 145 degrees is defined between a first ray and a second ray, the first ray being defined between the first point and a vertex, and the second ray being defined between the second point and the vertex, wherein the vertex is located at the bottom of the open channel and intersects the center line.

In any of the above embodiments, the snare wire includes a first end and a second end opposing the first end, and the first end and the second end are connected to the shaft with a crimp band.

In any of the above embodiments, the snare wire is formed of at least one or more of the following materials: stainless steel, titanium, a titanium alloy, and a cobalt alloy.

In any of the above embodiments, the overall length of the snare wire from the first end to the second end is in the range of 40 millimeters to 120 millimeters.

In any of the above embodiments, the loop exhibits a snare length in the range of 20 millimeters to 40 millimeters and a snare width in the range of 10 millimeters to 20 millimeters.

In any of the above embodiments, the loop exhibits a configuration selected from the following group of configurations: oval, rounded, elliptical, and polygonal shapes.

In any of the above embodiments, the open channel exhibits at least one of a curvate geometry and a polygonal geometry.

In any of the above embodiments, the snare wire consists of a single snare wire.

In any of the above embodiments, the snare further includes an actuator. The actuator includes a thumb loop mounted on a stem connected to the sheath and a finger loop connected to a side of a tube defining a bore extending the length of the tube. The stem is slidably disposed in the bore, and the tube is connected to the shaft.

According to various aspects, the present disclosure is directed to a method of assembling a snare. The method includes forming a loop in a snare wire, wherein the snare wire includes an exterior surface, and the snare wire exhibits a length and a cross-section, wherein the cross-section is orthogonal to the length, a groove extends helically along the length of the snare wire, wherein the groove is defined by an open channel in the cross-section, the open channel including an opening defined at the exterior surface between a first point and a second point, wherein the cross-section defines a center line and a diameter, wherein the open channel exhibits a groove depth less than half of the diameter of the cross-section, wherein the open channel is bound in the cross-section by a first line and a second line, the first line intersects the first point and the second line intersects the second point, the first line and the second line are parallel to the center line, and the first line and the second line are equidistant from the center line. The method further includes connecting the loop to a shaft; and slidably disposing the shaft in a sheath.

According to various aspects, the present disclosure is directed to a method of resecting a polyp. The method includes positioning a loop of a snare wire around a polyp, wherein the snare wire is connected to a shaft and the shaft is slidably disposed in a sheath, and the snare wire includes an exterior surface, and the snare wire exhibits a length and a cross-section, wherein the cross-section is orthogonal to the length, a groove extends helically along the length of the snare wire, wherein the groove is defined by an open channel in the cross-section, the open channel including an opening defined at the exterior surface between a first point and a second point, wherein the cross-section defines a center line and a diameter, wherein the open channel exhibits a groove depth less than half of the diameter of the cross-section, wherein the open channel is bound in the cross-section by a first line and a second line, the first line intersects the first point and the second line intersects the second point, the first line and the second line are parallel to the center line, and the first line and the second line are equidistant from the center line. The method further includes retracting the snare wire into the sheath and resecting the polyp with the snare wire.

### BRIEF DESCRIPTION OF DRAWINGS

The drawings described herein are for illustration purposes only and are not intended to limit the scope of the present disclosure in any way.

FIG. 1 illustrates a polypectomy snare according to embodiments of the present disclosure.

FIG. 2 illustrates a snare wire extending from a distal end of a snare according to embodiments of the present disclosure.

FIG. 3 illustrates a snare wire according to embodiments of the present disclosure.

FIG. 4A illustrates a cross-section of a snare wire according to embodiments of the present disclosure.

FIG. 4B illustrates the cross-section of a snare wire of FIG. 4A according to embodiments of the present disclosure.

FIG. 5A illustrates a cross-section of a snare wire according to embodiments of the present disclosure.

FIG. 5B illustrates the cross-section of a snare wire of FIG. 5A according to embodiments of the present disclosure.

FIG. 6A illustrates a cross-section of a snare wire according to embodiments of the present disclosure.

FIG. 6B illustrates the cross-section of a snare wire of FIG. 6A according to embodiments of the present disclosure.

FIG. 7A illustrates a cross-section of a snare wire according to embodiments of the present disclosure.

FIG. 7B illustrates the cross-section of a snare wire of FIG. 7A according to embodiments of the present disclosure.

FIG. 8 illustrates a method of resecting a polyp according to embodiments of the present disclosure.

FIG. 9 illustrates a method of forming a snare according to embodiments of the present disclosure.

### DETAILED DESCRIPTION

The following description is merely exemplary in nature and is not intended to limit the present disclosure, application, or uses. Furthermore, there is no intention to be bound by any expressed or implied theory presented in the preceding introduction, summary, or the following detailed description. It should be understood that throughout the drawings, corresponding reference numerals indicate like or corresponding parts and features.

Reference will now be made in detail to several examples of the disclosure that are illustrated in accompanying drawings. Whenever possible, the same or similar reference numerals are used in the drawings and the description to refer to the same or like parts or steps. The drawings are in simplified form and are not to precise scale.

The present disclosure relates to a snare including a snare wire with a helical groove, a method of forming the snare, and a method of resecting a polyp using the snare. The present disclosure further relates to a method of deploying the snare for resecting tissue. While the present technology is described primarily herein in connection with cold polypectomy snares used in endoscopic procedures, such as colonoscopies, the technology is not limited to cold polypectomy snares used in colonoscopies but may alternatively be employed in other types of snares and in performing other procedures such as the removal of nasal polyps, the removal of car polyps, gastrointestinal endoscopic mucosal resection, and hysteroscopies.

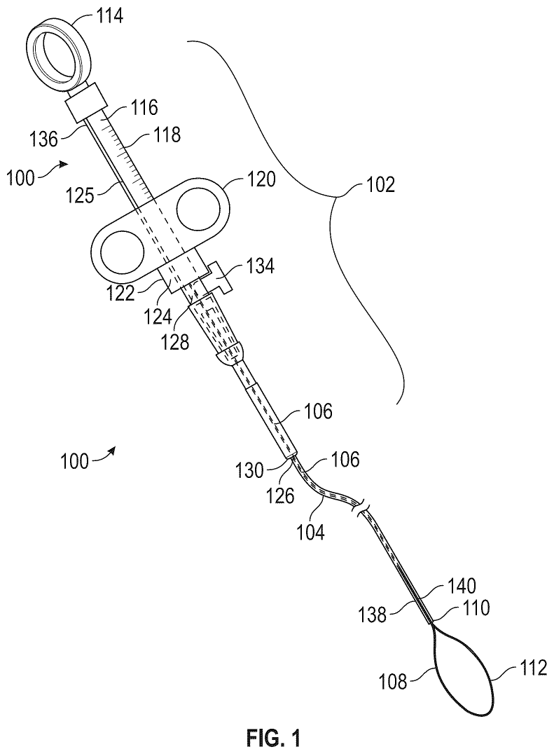

FIG. 1 illustrates an embodiment of a snare 100 including an actuator 102, a sheath 104, a shaft 106, and a snare wire 108 coupled to the shaft 106. The actuator 102, which is optional in embodiments, moves the shaft 106 and the snare wire 108 back and forth relative to the distal end 110 of the sheath 104. In embodiments, the actuator 102 includes a thumb loop 114 mounted on a proximal end 116 of a stem 118 and finger loops 120 that are movable relative to the thumb loop 114 and stem 118. As illustrated, the finger loops 120 are connected to a tube 122 and are arranged in a plane extending from either side of the tube 122. While two finger loops 120 are illustrated, it should be appreciated that a single finger loop 120 may be provided or more than two finger loops 120 may be provided. The tube 122 defines a bore 124 extending through the tube 122. The stem 118 passes through the tube 122 in the bore 124 and the tube 122 is movable relative to the stem 118 allowing the finger loops 120 to move relative to the stem 118 and thumb loop 114. In addition, the stem 118 is hollow and includes a slot 125 providing access to the interior of the stem 118.

The proximal end 126 of the sheath 104 is connected to the distal end 130 of the stem 118 and defines a lumen 132 therein. The sheath 104 is formed from at least one of a metal, metal alloy, and a polymer. At least a portion of the shaft 106 is slidably disposed in the lumen 132 of the sheath 104. The proximal end 128 of the shaft 106 also extends into the stem 118 and is connected to the tube 122. The snare wire 108 is an elongate wire formed into a loop 112 and connected to a distal end 138 of the shaft 106 by a weld or crimp 140.

As the finger loops 120 are advanced towards the distal end 130 of the stem 118 at least a portion of the loop 112 of the snare wire 108 and, in some embodiments, the shaft 106, extends out of the distal end 110 of the sheath 104. In embodiments, the finger loops 120 may be advanced to a first stop 134, which prohibits over travel of the finger loops 120 relative to the stem 118. When the finger loops 120 are retracted and positioned at a proximal end 116 of the stem 118 adjacent to the thumb loop 114, the snare wire 108 is fully retracted within the sheath 104. Alternatively, in embodiments where an actuator 102 is not present, the shaft 106 is manipulated relative to the sheath 104 to slidably displace the shaft 106 within the sheath 104 and to extend and retract the snare wire 108 from the distal end 110 of the sheath 104.

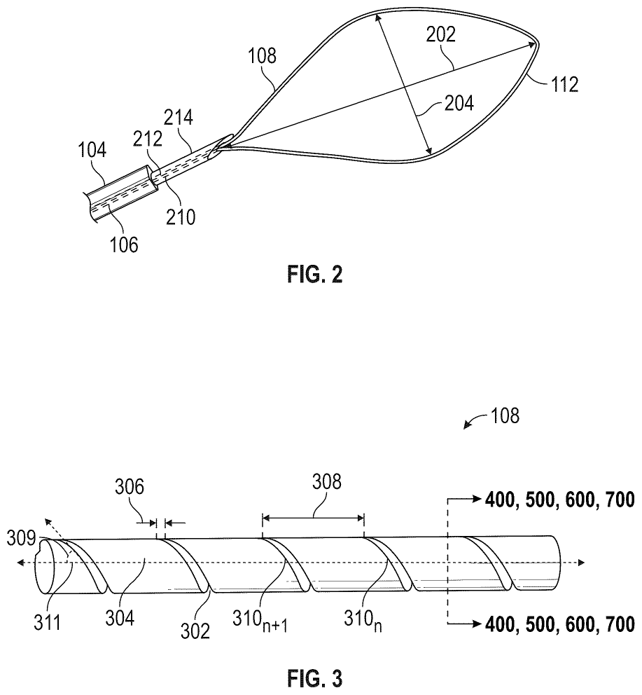

FIG. 2 illustrates a close-up of the snare wire 108. As noted above, the snare wire 108 is formed into a loop 112. In embodiments, the loop 112 is formed by folding the snare wire 108 back on itself such that a first end 210 of the snare wire 108 is affixed to itself a given length from the first end 210, between the first end 210 and the second end 212 opposing the first end 210. Alternatively, the first end 210 of the snare wire 108 is affixed to the second end 212 of the snare wire 108 as illustrated in FIG. 2. The loop 112 is affixed in place by, at least one of twisting and crimping the snare wire 108. In embodiments, a crimp band 214, such as a metal tube, or a heat shrink band, may be used to secure the loop 112 by placing the crimp band 214 and crimping the crimp band 214 over the first end 210 and either the second end 212 or the first end 210 and a point between the first end 210 and the second end 212. Additionally, in embodiments, the crimp band 214 may also be used to secure at least the second end 212 and, in some embodiments, the first end 210 of the snare wire 108 to the shaft 106. The loop 112 may take on a number of configurations including rounded, oval, elliptical and angular polygonal shapes. In FIG. 2, the loop 112 is illustrated as oval; however, the loop 112 may also assume the configuration of, but not limited to, a hexagon, diamond, rhombus, or a duckbill.

The snare wire 108 is formed from at least one of stainless steel, titanium, a titanium alloy, and a cobalt alloy. In embodiments, the snare wire 108 is formed from one or more of the following materials: stainless steel 304V, stainless steel 316 LVM, steel 35N LT (available from Fort Wayne Metals), nickel titanium alloys such as nitinol, FWM 1058 (available from Fort Wayne Metals), and titanium. The snare wire 108 is manipulatable and shapable, and may be used repeatedly during a procedure due (at least in part) to the materials used for the snare wire 108. The snare wire 108 exhibits an overall length in the range of 40 millimeters to 120 millimeters, including all values and ranges therein. The overall length is understood as the length of the snare wire 108 from the first end 210 to the second end 212. Further, the snare loop 112 exhibits a snare length 202 in the range of 20 millimeters to 40 millimeters, including all values and ranges therein such as 30 millimeters plus or minus 10 millimeters, and a snare width 204 in the range of 10 millimeters to 20 millimeters, including all values and ranges therein such as 15 millimeters plus or minus 5 millimeters.

Turning FIG. 3, the figure illustrates a close-up view of the snare wire 108. In the illustrated embodiment, the snare wire 108 is formed from only a single wire. In alternative embodiments, more than one snare wire 108 may be present. The snare wire 108 includes a groove 302 formed in the exterior surface 304 of the snare wire 108 that wraps helically around the snare wire 108 along the length of the snare wire 108. The groove 302 exhibits a helix pitch 308, which is understood as the distance between a point on the leading edge of a first wrap 310n and the corresponding point on the leading edge of a second, adjacent wrap 310n+1. In embodiments, the helix pitch 308 remains the same along the entire length of the snare wire 108. In alternative embodiments, the helix pitch 308 can vary. The helix pitch 308 is in the range of 2.0 millimeters to 3.0 millimeters, including all values and ranges therein, such as 2.5 millimeters. In addition, the helix pitch 308 may vary between a first wrap 310n and an adjacent wrap 310n+1 by plus or minus 0.5 millimeters, including all values and ranges therein, such as plus or minus 3 millimeters. The groove 302 also exhibits a helix angle 309. which is understood as the angle between the helix formed by the groove 302 and the center axis 311 of the snare wire 108. In embodiments, the helix angle 309 is in the range of 15 degrees to 75 degrees, including all values and ranges therein.

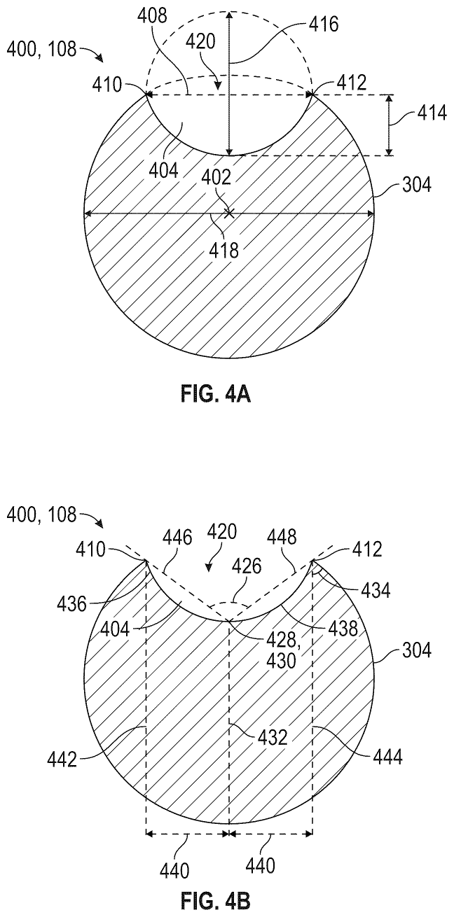

FIGS. 4A and 4B illustrate a cross-section 400 of the snare wire 108 including the helical groove 302 of the snare wire 108. The cross-section 400 is defined at 90 degrees to, or orthogonal to, the axial axis, which is generally defined by the length of the snare wire 108 as illustrated by cross-section 400, 500, 600, 700 illustrated in FIG. 3. As illustrated, the snare wire 108 exhibits a round cross-section 400. In alternative embodiments, the snare wire 108 may exhibit other curvate geometries and be oblong, elliptical, or the snare wire 108 may exhibit a polygonal geometry exhibiting three or more segments.

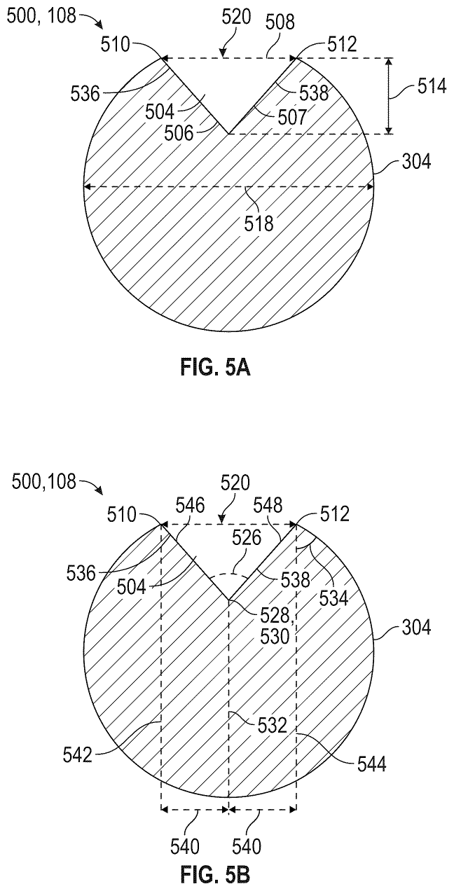

The groove 302 defines, or is formed by, an open channel 404 in the exterior surface 406 of the snare wire 108. In the illustrated embodiment, the open channel 404 is concave. In alternative embodiments, some of which are illustrated in FIGS. 5 through 7, the open channel 404 defines other geometries including rounded and polygonal geometries including two or more segments such as in the range of 2 segments to 10 segments. Progressing along the length of the snare wire 108, the open channel 404 rotates relative to the center 402 of the cross-section 400. With reference to FIG. 4A, the groove width 408, measured between two points 410, 412 defining the opening 420 of the open channel 404 in the exterior surface 304, is in the range of 0.05 millimeters to 1.5 millimeters, including all values and ranges therein, such as 1.0 millimeters. The groove depth 414, measured between the deepest point in the groove 302 and the opening 420 of the open channel 404, is in the range of 0.04 millimeters to 0.06 millimeters, including all values and ranges therein such as 0.053 millimeters. The groove diameter 416 is in the range of 0.2 to 0.3 millimeters, including all values and ranges therein, such as 0.248 millimeters. The cross-sectional diameter 418, or the cross-sectional length, of the snare wire 108 is in the range of 0.2 millimeters to 0.8 millimeters, including all values and ranges therein such as 0.254 millimeters to 0.762 millimeters. In the case of non-circular cross-sections (as well as circular cross-sections 400), the cross-sectional length 418 is understood herein as the longest distance between any two points on the periphery of the cross-section 400. The groove depth 414 extends less than half of the cross-sectional length 418 of the snare wire 108.

Turning now to FIG. 4B, the opening 420 is defined between the two points 410, 412. In embodiments, the exterior surface 304 and each side 436, 438 of the open channel 404 adjacent to the exterior surface 304 meet at an acute angle 434 of less than 90 degrees, such as in the range of 30 degrees to 89 degrees, including all values and ranges therein. In alternative embodiments, the two points 410, 412 exhibit a chamfer or a corner having a radius. In such embodiments, the two points 410, 412 are determined by the intersection of lines extended from each side 436, 438 of the open channel 404 and the exterior surface 304.

In addition, the snare wire 108 defines a center line 432 and the two points 410, 412 are equidistant a distance 440 from the center line 432. Further, the open channel 404 is bound on either side by two lines 442, 444. Each line 442, 444 intersects one of the two points 410, 412 and runs parallel to the center line 432, i.e., the first line 442 intersects the first point 410 and the second line 444 intersects the second point 412. In addition, a first ray 446 is defined between the first point 410 and the bottom 430 of the open channel 404 where it intersects the center line 432, and a second ray 448 is defined between the second point 412 and the bottom of the open channel 404 where it intersects the center line 432. An angle 426 is defined between the two rays 446, 448 and the vertex 428 at the bottom 430 of the open channel 404 intersecting the center line 432. The angle 426 is in the range of 45 degrees to 145 degrees, including all values and ranges therein.

FIGS. 5A and 5B illustrate another embodiment of a cross-section 500 of the snare wire 108. The helical groove 302 is formed by the open channel 504, which exhibits a configuration having a generally polygonal shape including two segments 506, 507. With reference to FIG. 5A, the groove width 508, measured between two points 510, 512 at the opening 520 of the open channel 504 where the open channel 504 is defined in the exterior surface 304, is in the range of 0.05 millimeters to 1.5 millimeters, including all values and ranges therein, such as 1.0 millimeters. The groove depth 514, measured between the deepest point of the groove 302 and the opening 520 of the open channel 504, is in the range of 0.04 millimeters to 0.06 millimeters, including all values and ranges therein such as 0.053 millimeters. The cross-sectional diameter 518, or the cross-sectional length, of the snare wire 108 is in the range of 0.2 millimeters to 0.8 millimeters, including all values and ranges therein such as 0.254 millimeters to 0.762 millimeters. The groove depth 514 extends less than half of the cross-sectional length 518 of the snare wire 108. As described above with respect to FIG. 4A, in the case of non-circular cross-sections (as well as circular cross-sections 500), the cross-sectional length 518 is understood as the longest distance between any two points on the periphery of the cross-section 500.

Turning now to FIG. 5B, the opening 520 is defined between the two points 510, 512. In embodiments, the exterior surface 304 and each side 536, 538 of the open channel 504 meet at an acute angle 534 of less than 90 degrees, such as in the range of 30 degrees to 89 degrees, including all values and ranges therein. In alternative embodiments, the two points 510, 512 exhibit a chamfer or a corner having a radius. In such embodiments, the two points 510, 512 are determined by the intersection of lines extended from each side 536, 538 of the open channel 504 adjacent to the exterior surface 304 and the exterior surface 304.

Again, as with FIG. 4B, the snare wire 108 defines a center line 532 and the two points 510, 512 are equidistant a distance 540 from the center line 532. Further, the open channel 504 is bound on either side by the two lines 542, 544. The first line 542 intersects the first point 510 and the second line 544 intersects the second point 512 and the two lines 542, 544 run parallel to the center line 532. In addition, a first ray 546 is defined between the first point 510 and the bottom 530 of the open channel 504 where it intersects the center line 532, and a second ray 548 is defined between the second point 512 and the bottom 530 of the open channel 504 where it intersects the center line 532. An angle 526 is defined between the two rays 546, 548 and the vertex 528 at the bottom 530 of the open channel 504 intersecting the center line 532. The angle 526 is in the range of 45 degrees to 145 degrees, including all values and ranges therein.

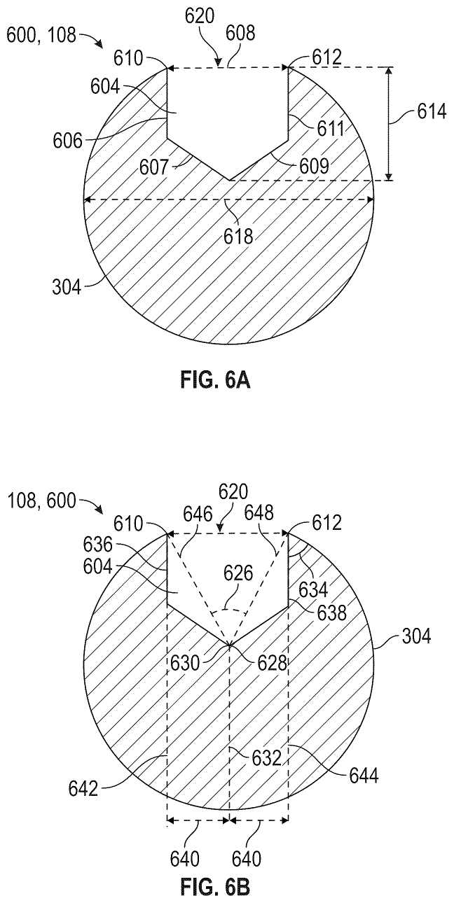

FIGS. 6A and 6B illustrate another embodiment of a cross-section 600 of the snare wire 108. In this embodiment, the groove 302 is formed by the open channel 604, which assumes a polygonal shape including four segments 606, 607, 609, 611. With reference to FIG. 6A, the groove width 608 is measured between two points 610, 612 at the opening 620 of the open channel 604 where the open channel 604 is defined in the exterior surface 304, and is in the range of 0.05 millimeters to 1.5 millimeters, including all values and ranges therein, such as 1.0 millimeters. The groove depth 614, measured between the deepest point of the groove 302 and the opening 620 of the open channel 604, is in the range of 0.04 millimeters to 0.06 millimeters, including all values and ranges therein such as 0.053 millimeters. The cross-sectional diameter 618, or the cross-sectional length, of the snare wire 108 is in the range of 0.2 millimeters to 0.8 millimeters, including all values and ranges therein such as 0.254 millimeters to 0.762 millimeters. Again, the groove depth 614 extends less than half of the cross-sectional length 618 of the snare wire 108. As described above with respect to FIGS. 4A and 5A, in the case of non-circular cross-sections (as well as circular cross-sections 600), the cross-sectional length 618 is understood as the longest distance between any two points on the periphery of the cross-section 600.

Turning now to FIG. 6B, the opening 620 is defined between the two points 610, 612. In embodiments, the exterior surface 304 and each side 636, 638 of the open channel 604 meet at an acute angle 634 of less than 90 degrees such as in the range of 30 degrees to 60 degrees, including all values and ranges therein. In alternative embodiments, the two points 610, 612 exhibit a chamfer or a corner having a radius. In such embodiments, the two points 610, 612 are determined by the intersection of lines extended from each side 636, 638 of the open channel 604 adjacent to the exterior surface 304 and the exterior surface 304.

Again, as with FIGS. 4B and 5B, the snare wire 108 defines a center line 632 and the two points 610, 612 are equidistant a distance 640 from the center line 632. Further, the open channel 604 is bound on either side by the two lines 642, 644. Each line 642, 644 intersects one of the two points 610, 612 and runs parallel to the center line 632, i.e., the first line 642 intersects the first point 610 and the second line 644 intersects the second point 612. In addition, a first ray 646 is defined between the first point 610 and the bottom 630 of the open channel 604 where it intersects the center line 632, and a second ray 648 is defined between the second point 612 and the bottom 630 of the open channel 604 where it intersects the center line 632. An angle 626 is defined between the two rays 646, 648 and the vertex 628 at the bottom 630 of the open channel 604 intersecting the center line 632. The angle 626 is in the range of 45 degrees to 145 degrees, including all values and ranges therein.

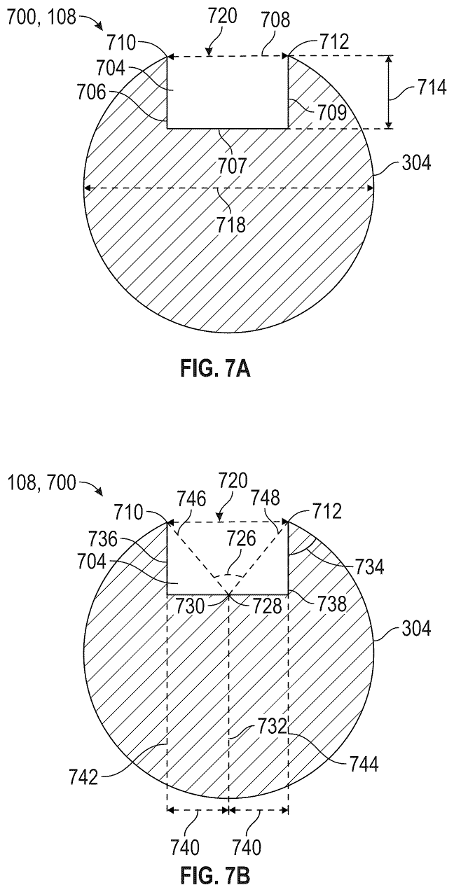

FIGS. 7A and 7B illustrate another embodiment of a cross-section 700 of the snare wire 108. In this embodiment, the groove 302 the groove 302 is formed by an open channel 704 assuming a polygonal shape including three segments 706, 707, 709. With reference to FIG. 7A, the groove width 708 is measured between two points 710, 712 at the opening 720 of the open channel 704 where the open channel 704 is defined in the exterior surface 304, and is in the range of 0.05 millimeters to 1.5 millimeters, including all values and ranges therein, such as 1.0 millimeters. The groove depth 714, measured between the deepest point of the groove 302 and the opening 720 of the open channel 704, is in the range of 0.04 millimeters to 0.06 millimeters, including all values and ranges therein such as 0.053 millimeters. The cross-sectional diameter 718, or the cross-sectional length, of the snare wire 108 is in the range of 0.2 millimeters to 0.8 millimeters, including all values and ranges therein such as 0.254 millimeters to 0.762 millimeters. The groove depth 714 extends less than half of the cross-sectional length 718 of the snare wire 108. As described above with respect to FIGS. 4A, 5A, and 6A, in the case of non-circular cross-sections (as well as circular cross-sections 700), the cross-sectional length 718 is understood as the longest distance between any two points on the periphery of the cross-section 700.

Turning now to FIG. 7B, the opening 720 is defined between the two points 710, 712. In embodiments, the exterior surface 304 and each side 736, 738 of the open channel 704 adjacent to the exterior surface 304 and the exterior surface 304 meet at an acute angle 734of less than 90 degrees such as in the range of 30 degrees to 60 degrees, including all values and ranges therein. In embodiments, the two points 710, 712 exhibit a chamfer or a corner 734 having a radius. In such embodiments, the two points 710, 712 are determined by the intersection of lines extended from each side 736, 738 and the exterior surface 304.

And again, as with FIGS. 4B, 5B and 6B, the snare wire 108 defines a center line 732 and the two points 710, 712 are equidistant a distance 740 from the center line 732. Further, the open channel 704 is bound on either side by the two lines 742, 744. Each line 742, 744 intersects one of the two points 710, 712 and runs parallel to the center line 732. In addition, a first ray 746 is defined between the first point 710 and the bottom 730 of the open channel 704 where it intersects the center line 732, and a second ray 748 is defined between the second point 712 and the bottom 730 of the open channel 704 where it intersects the center line 732. An angle 726 is defined between the two rays 746, 748 and the vertex 728 at the bottom 730 of the open channel 704 intersecting the center line 732. The angle 726 is in the range of 45 degrees to 145 degrees, including all values and ranges therein.

In any of the above embodiments, the cross-sectional area of the open channel 404, 504, 604, 704 overlapping the total cross-sectional area of the snare wire 108 is in the range of 5 percent to 20 percent, including all values and ranges therein, of the total cross-sectional area of the snare wire 108. Further, while the open channels 404, 504, 604, 704 are illustrated as being mirrored around the center line 432, 532, 632, 732, in alternative embodiments, the open channel may exhibit an irregular geometry that that is not mirrored around the center line. It should be appreciated that alternative open channel geometries in addition to those exhibited in FIGS. 4A through 7B similarly exhibit the features described above with reference to FIGS. 4A through 7B.

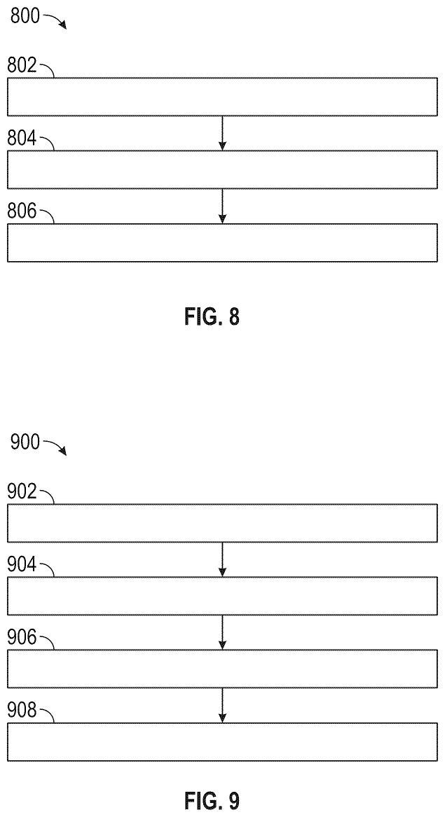

FIG. 8 illustrates a method 800 of using a snare 100 according to any of the above described embodiments for resecting a polyp. With further reference to FIGS. 1 through 7 at block 802 a loop 112 of a snare wire 108 is positioned around a polyp. At block 804 the snare wire 108 is then retracted into the sheath 104 of the snare 100 by sliding the shaft 106 relative to the sheath 104. In embodiments including an actuator 102, the snare wire 16 is retracted into the sheath 104 by moving the thumb loop 114 and finger loops 120 of an actuator 102 closer together. During retraction a portion of the tissue surrounding the polyp is received and held within the groove 302, preventing slippage of the snare wire 108 relative to the polyp. At block 806 the polyp is resected by the snare wire 108 and removed from the mucosal membrane to which the polyp was attached.

The present disclosure also includes a method 900 of forming a snare 100, an embodiment of which is illustrated in FIG. 9, described with reference to FIGS. 1 through 7. The method includes at block 902 forming a loop 112 in a snare wire 108 according to any of the above described embodiments. The loop 112 is connected to the shaft 106 at block 904 as described above. In embodiments, the loop 112 is secured prior to connecting the loop 112 to the shaft 106 or when the loop 112 is connected to the shaft 106 using a crimp band 214 as noted above. At block 906 the shaft 106 is slidably disposed in a sheath 104. Optionally, at block 908, the sheath 104 is connected to the stem 118 of an actuator 102 according to any of the above described embodiments, and the shaft 106 is connected to the tube 122 of the actuator 102.

A study was performed comparing two competitive, commercially available snares, Competitive Snare A and Competitive Snare B, to a snare 100 including a snare wire 108 as illustrated in FIGS. 4A and 4B. Four samples of each snare were each used to make five cuts. Sixty polyps were resected in total. Competitive snare A included a round snare shape having a 15 millimeter loop width and a braided snare wire. Competitive snare B included a hexagonal snare shape with a 15 millimeter loop width and a 0.3 millimeter wire diameter. The snare 100 of including a snare wire 108 of FIGS. 4A and 4B was cut to 90 millimeters in length and both ends were crimped to the end of a handle guide wire using a small metal tube of approximately 5 millimeters in length. The snare wire was formed into an oval shape. The snare 100 including the above described snare wire 108 as illustrated in FIGS. 4A and 4B exhibited a 23% increase in the resection site area compared to the braided round snare and a 29% increase in the resection site area compared to the seven-wire braided hexagonal snare wire.

The snares and methods described herein offer a number of advantages. These advantages include, for example, the ability of the tissue to settle into the groove during a resection decreasing slippage of the snare wire relative to the polyp. In addition, these advantages include providing multiple points of contact with tissue during resection due to the helical shape of the groove, which also allows for the snare wire to be secured to the snare without undue concern for the orientation of the snare wire. These advantages also include the ability of the snare wire to anchor on to and capture a relatively larger surface area of tissue. These advantages yet also include the ability to resect polyps having a diameter in the range of 1 millimeter to 40 millimeters. These advantages further include the ability to manipulate the shape of the snare wire and reshape the snare wire if needed as well as the ability to use the snare wire multiple times during a procedure, due (at least in part) to the materials employed. These advantages also include a reduction in the likelihood of piecemeal extraction. These advantages further include a reduction in snare slippage over a lesion.

The description of the present disclosure is merely exemplary in nature and variations that do not depart from the gist of the present disclosure are intended to be within the scope of the present disclosure. Such variations are not to be regarded as a departure from the spirit and scope of the present disclosure.

What is claimed is:

1. A snare, comprising:
a sheath defining a lumen, the sheath including a proximal end and a distal end;
a shaft slidably disposed in the sheath;
a snare wire coupled to the shaft, wherein the snare wire is formed into a loop, the snare wire includes an exterior surface, and the snare wire exhibits a length and a cross-section, wherein the cross-section is orthogonal to the length; and
a groove extending helically along the length of the snare wire, wherein the groove is defined by an open channel in the cross-section, the open channel includes an opening defined at the exterior surface between a first point and a second point,
wherein the cross-section defines a center line and a cross-sectional length,
wherein the open channel exhibits a groove depth less than half of the cross-sectional length, and
wherein the open channel is bound in the cross-section by a first line and a second line, the first line intersects the first point and the second line intersects the second point, the first line and the second line are parallel to the center line, and the first line and the second line are equidistant from the center line.

2. The snare of claim 1, wherein the cross-section exhibits a cross-sectional diameter in the range of 0.2 millimeters to 0.8 millimeters.

3. The snare of claim 1, wherein the groove defines a helix pitch along the length of the snare wire and the helix pitch is in the range of 2 millimeters to 3 millimeters.

4. The snare of claim 1, wherein the cross-section defines a total cross-sectional area and the open channel defines a second cross-sectional area overlapping the total cross-sectional area, wherein the second cross-sectional area is in the range of 5 percent to 20 percent of the total cross-sectional area.

5. The snare of claim 1, wherein the open channel exhibits a geometry that is mirrored around the center line.

6. The snare of claim 1, wherein the groove width is in the range of 0.5 millimeters to 1.5 millimeters.

7. The snare of claim 1, wherein the groove depth is in the range of 0.04 millimeters to 0.06 millimeters.

8. The snare of claim 1, wherein the cross-section of the groove is concave and exhibits groove diameter in the range of 0.2 millimeters to 0.3 millimeters.

9. The snare of claim 1, wherein the exterior surface and a first surface of the open channel adjacent to the exterior surface meet at the first point at an acute angle and the exterior surface and a second surface of the open channel adjacent to the exterior surface meet at the second point at an acute angle.

10. The snare of claim 1, wherein the open channel includes a bottom and wherein an angle in the range of 45 degrees to 145 degrees is defined between a first ray and a second ray, the first ray being defined between the first point and a vertex, and the second ray being defined between the second point and the vertex, wherein the vertex is located at the bottom of the open channel and intersects the center line.

11. The snare of claim 1, wherein the snare wire includes a first end and a second end opposing the first end, and the first end and the second end are connected to the shaft with a crimp band.

12. The snare of claim 1, wherein the snare wire is formed of at least one or more of the following materials: stainless steel, titanium, a titanium alloy, and a cobalt alloy.

13. The snare of claim 1, wherein the overall length of the snare wire from the first end to the second end is in the range of 40 millimeters to 120 millimeters.

14. The snare of claim 1, wherein the loop exhibits a snare length in the range of 20 millimeters to 40 millimeters and a snare width in the range of 10 millimeters to 20 millimeters.

15. The snare of claim 1, wherein the loop exhibits a configuration selected from the following group of configurations: oval, rounded, elliptical, and polygonal shapes.

16. The snare of claim 1, wherein the open channel exhibits at least one of a curvate geometry and a polygonal geometry.

17. The snare of claim 1, wherein snare wire consists of a single snare wire.

18. The snare of claim 1, further comprising: an actuator wherein the actuator includes a thumb loop mounted on a stem connected to the sheath and a finger loop connected to a side of a tube defining a bore extending the length of the tube, wherein the stem is slidably disposed in the bore, and the tube is connected to the shaft.

19. A method of assembling a snare, comprising:
forming a loop in a snare wire, wherein the snare wire includes an exterior surface, and the snare wire exhibits a length and a cross-section, wherein the cross-section is orthogonal to the length, a groove extends helically along the length of the snare wire, wherein the groove is defined by an open channel in the cross-section, the open channel including an opening defined at the exterior surface between a first point and a second point, wherein the cross-section defines a center line and a diameter, wherein the open channel exhibits a groove depth less than half of the diameter of the cross-section, wherein the open channel is bound in the cross-section by a first line and a second line, the first line intersects the first point and the second line intersects the second point, the first line and the second line are parallel to the center line, and the first line and the second line are equidistant from the center line;
connecting the loop to a shaft; and
slidably disposing the shaft in a sheath.

20. A method of resecting a polyp, comprising:
positioning a loop of a snare wire around a polyp, wherein the snare wire is connected to a shaft and the shaft is slidably disposed in a sheath, and the snare wire includes an exterior surface, and the snare wire exhibits a length and a cross-section, wherein the cross-section is orthogonal to the length, a groove extends helically along the length of the snare wire, wherein the groove is defined by an open channel in the cross-section, the open channel including an opening defined at the exterior surface between a first point and a second point, wherein the cross-section defines a center line and a diameter, wherein the open channel exhibits a groove depth less than half of the diameter of the cross-section, wherein the open channel is bound in the cross-section by a first line and a second line, the first line intersects the first point and the second line intersects the second point, the first line and the second line are parallel to the center line, and the first line and the second line are equidistant from the center line;
retracting the snare wire into the sheath; and
resecting the polyp with the snare wire.
